(19) United States
(12) Patent Application Publication
(10) Pub. No.: US 2009/0104194 A1
(43) Pub. Date: Apr. 23, 2009

(54) BI-DIRECTIONAL ANTIBODY FOR TREATMENT OF OCULAR DISORDERS

(76) Inventor: Richard A. Schatz, San Diego, CA (US)

Correspondence Address: Neil K. Nydegger, NYDEGGER & ASSOCIATES, 348 Olive Street, San Diego, CA, 92103, US

(21) Appl. No.: 11/876,124

(22) Filed: Oct. 22, 2007

(51) Int. Cl. A61K 39/395; A61P 27/02

(52) U.S. Cl. 424/136.1

(57) ABSTRACT

A system and method for treating ocular disorders are disclosed. Specifically, the system and method administer bi-directional antibodies to treat a retina. Structurally, each bi-directional antibody has a first receptor arm and a second receptor arm. Further, each first receptor arm possesses binding affinity for retinal cells and each second receptor arm possesses binding affinity for melanin. After the antibodies are administered to the retina, the first receptor arms attach to the retinal cells. Thereafter, melanin is delivered to the retina and the second receptor arms are fixed to the melanin. In this manner, melanin is concentrated at the retinal cells.

### FIELD OF THE INVENTION

The present invention pertains generally to processes for binding medicaments to cells. More particularly, the present invention pertains to a system and method for treating ocular disorders with medicaments. The present invention is particularly, but not exclusively, useful as a system and method for utilizing bidirectional antibodies as selective binding agents in treating retinas with medicaments.

### BACKGROUND OF THE INVENTION

Ocular albinism is an inherited condition in which the eyes lack melanin pigment. More specifically, the eyes include pigment-making cells (melanocytes) but, because of an inborn error of metabolism, the melanocytes cannot form melanin. As a result of the lack of pigment in the eyes, various vision problems are exhibited. For instance, persons with ocular albinism typically experience reduced visual acuity from 20/60 to 20/400, nystagmus (the involuntary back-and-forth movement of the eyes), strabismus (crossed eyes or “lazy” eye), and/or photophobia (sensitivity to bright light and glare). Further, these symptoms may result in difficulty in school, such as inability to read what is written on a chalkboard except when very close, difficulty with sports, and an inability to operate automobiles and other machinery.

In ocular albinism, the main problem with the eye is in the fovea, a small area of the retina which affords acute vision. Specifically, it has been determined that, for those with ocular albinism, the fovea does not develop completely. This failure to develop presumably occurs because the melanin needed for the growth processes that normally occur is deficient. As a result, the eye cannot process sharp light images. Because the fovea is not developed well, it is difficult to correct vision completely with glasses for those with ocular albinism.

The disorder occurs in varying degrees of hypopigmentation, with total albinism exhibiting easily recognized signs while partially affected cases are much more difficult to detect. While studies in regard to the prevalence of albinism in one form or another vary greatly, estimates range upwards from one in 2,000 persons (partial albinism) to one in 16,000 persons in the United States (total albinism). However, the number of afflicted cases may be much higher since many may not even realize they have the disorder or they may be trying to hide the fact.

Presently, there is no cure for ocular albinism. For some of those with ocular albinism, low-vision aids may improve vision. Among the major optical forms of low-vision aids often recommended for persons with albinism are bioptic telescopic spectacles or even simple magnifiers. While contact lenses can improve vision, frequently persons with ocular albinism are unable to tolerate their lenses, possibly due to the heightened tactile sensitivity and nystagmus common in this disorder. Further, persons with ocular albinism often opt not to wear sunglasses despite their sensitivity to light. This is due to the fact that sunglasses reduce their already low vision.

In light of the above, it is an object of the present invention to provide a system and method for treating ocular disorders, such as ocular albinism. Another object of the present invention is to provide a system and method for utilizing antibodies to target medicament at the retina. Still another object of the present invention is to provide a system and method for treating a retina with antibodies and melanin to alleviate the symptoms of ocular albinism. Another object of the present invention is to provide a system and method for selectively binding medicament to a targeted site in a retina. Yet another object of the present invention is to provide a system for treating ocular disorders that is simple to implement, easy to use, and comparatively cost effective.

### SUMMARY OF THE INVENTION

In accordance with the present invention, a system and method are provided for treating ocular disorders. More specifically, the system and method are provided for treating retinal disorders such as ocular albinism. For this purpose, the system utilizes bi-directional antibodies as selective binding agents to treat the retina of a patient suffering from the ocular disorder. Structurally, each bi-directional antibody has a first receptor arm and a second receptor arm. Further, each first receptor arm possesses binding affinity for retinal cells and each second receptor arm possesses binding affinity for melanin. Accordingly, the system includes melanin for treating the retina.

In operation, antibodies are administered to the retina. Specifically, the antibodies are administered via an intra retinal injection, an intra arterial injection, or an intra venous injection. Thereafter, the first receptor arms of the antibodies attach to the retina. After the antibodies attach to the retina, melanin is delivered to the retina through an intra retinal, an intra arterial, or an intra venous injection. Then the second receptor arms of the antibodies attach to the melanin. In this manner, melanin is targeted at the retina for treatment.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a cross sectional view of an eye of a patient suffering from an ocular disorder;

FIG. 2 is a schematic view of the retina of FIG. 1 treated with antibodies and a medicament in accordance with the present invention; and

FIG. 3 is a flow chart illustrating the method of the present invention.

### DESCRIPTION OF THE PREFERRED EMBODIMENTS

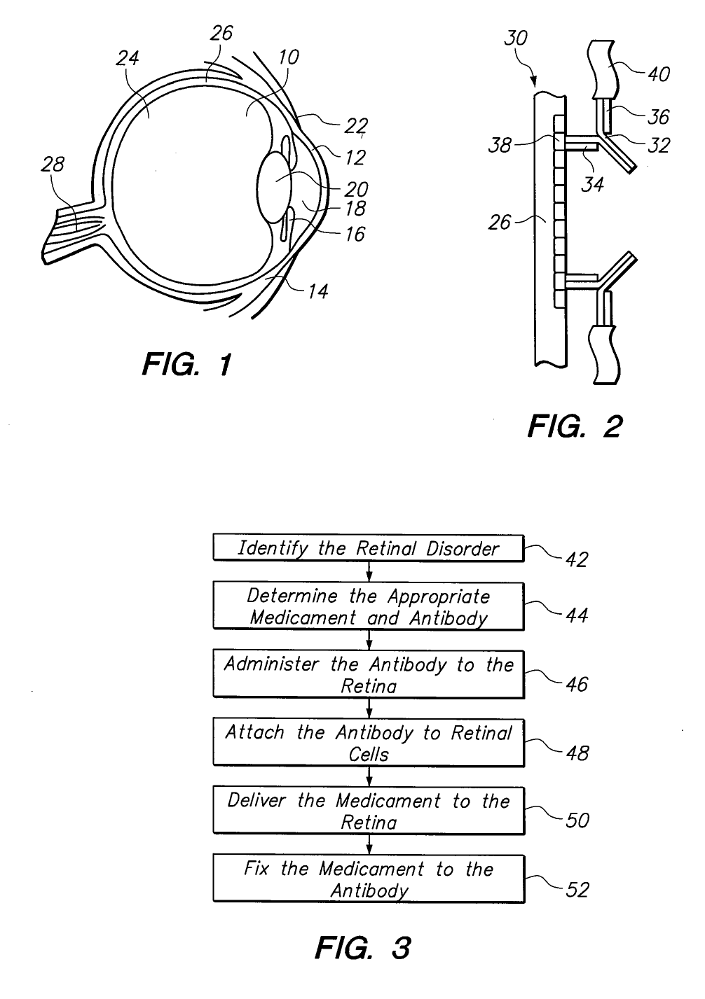

Referring initially to FIG. 1, the eye 10 of a patient suffering from an ocular disorder is illustrated. As shown, the eye 10 includes a cornea 12, sclera 14, iris 16, pupil 18 and lens 20 at its anterior 22. Extending from the anterior 22 to the posterior 24 of the eye 10 is the retina 26. As shown, the retina 26 is in communication with the retina artery 28 which supplies blood to the retina 26.

Referring now to FIG. 2, a system for treating a retina 26 is shown and generally designated 30. As shown, the system 30 includes bi-directional antibodies 32 which attach to the retina 26. Structurally, the antibodies 32 include first receptor arms 34 and second receptor arms 36. For purposes of the present invention, the first receptor arms 34 possess binding affinity for retinal cells 38. As a result, the first receptor arms 34 attach to the retinal cells 38, as shown. Further, the system 30 includes a medicament 40 which may comprise stem cells, melanin, or other proteins. For purposes of the present invention, the second receptor arms 36 possess binding affinity for the medicament 40. Accordingly, the second receptor arms 36 attach to the medicament 40 as shown. In this manner, the medicament 40 is concentrated at the retinal cells 38.

Turning to FIG. 3, the operation of the system 30 is illustrated. Initially, in action block 42, the ocular disorder suffered by the patient is identified. For instance, the ocular disorder may be a retinal disorder such as ocular albinism, in which a patient lacks sufficient melanin at the retina 26. After the ocular disorder is identified, the appropriate medicament 40 and antibody 32 is determined (action block 44). For ocular albinism, melanin is the appropriate medicament 40 and the antibody 32 is selected from a known group of antibodies which cooperate with retinal cells 38 and melanin 40. Thereafter, the antibodies 32 are administered to the retina 26 (action block 46). Specifically, the antibodies 32 may be administered through an intra retinal injection or an arterial injection into the retinal artery 28. Alternatively, the antibodies 32 may be administered retrogradely with a venous occlusion. After the antibodies 32 are received in the retina 26, their first receptor arms 34 attach to retinal cells 38 due to their binding affinity (action block 48). When the antibodies 32 have attached to the retinal cells 38, the medicament 40 is delivered to the retina 26 through an intra retinal or intra arterial injection (action block 50). Alternatively, the medicament 40 may be administered retrogradely with a venous occlusion. Thereafter, the medicament 40 is fixed to the second receptor arms 36 of the antibodies 32 (action block 52). As a result, the medicament 40 is concentrated at the retinal cells 38 for treatment of the retina 26.

While the particular Bi-directional Antibody for Treatment of Ocular Disorders as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

1. A method of treating an ocular disorder comprising the steps of:
administering bi-directional antibodies to a retina, wherein each bi-directional antibody has a first receptor arm and a second receptor arm, and wherein each first receptor arm possesses binding affinity for retinal cells and each second receptor arm possesses binding affinity for melanin;
attaching the first receptor arms to the retinal cells;
delivering melanin to the retina, with said melanin having receptor sites; and
fixing the second receptor arms to the receptor sites of the melanin to concentrate melanin at the retinal cells.

2. The method of claim 1 wherein the antibodies are administered to the retina by an intra retinal injection.

3. The method of claim 1 wherein the antibodies are administered to the retina by an intra arterial injection.

4. The method of claim 1 wherein the melanin is delivered to the retina by an intra retinal injection.

5. The method of claim 1 wherein the melanin is delivered to the retina by an intra arterial injection.

6. A method of treating ocular disorders comprising the steps of:
administering bi-directional antibodies to a retina, wherein each antibody possesses a first binding affinity for retinal cells;
attaching the antibodies to the retinal cells;
delivering a medicament to the retina, with said medicament having receptor sites, and wherein each antibody possesses a second binding affinity for the medicament; and
fixing the antibodies to the receptor sites of the medicament to concentrate the medicament at the retinal cells.

7. The method of claim 6 wherein the antibodies are administered to the retina by an intra retinal injection.

8. The method of claim 6 wherein the antibodies are administered to the retina by an intra arterial injection.

9. The method of claim 6 wherein the medicament is delivered to the retina by an intra retinal injection.

10. The method of claim 6 wherein the medicament is delivered to the retina by an intra arterial injection.

11. The method of claim 6 wherein the ocular disorder is ocular albinism and the medicament is melanin.

12. The method of claim 6 wherein the medicament is selected from a group comprised of stem cells, melanin, and proteins.

13. The method of claim 6 wherein each antibody has a first receptor arm and a second receptor arm, and wherein the first receptor arms of the antibodies attach to the retinal cells and the second receptor arms of the antibodies attach to the receptor sites of the medicament.

14. A system of treating ocular disorders comprising:
a medicament for treating retinal cells;
bi-directional antibodies having a first binding affinity for retinal cells and a second binding affinity for a medicament;
a means for administering the bi-directional antibodies to the retina;
a means for attaching the antibodies to the retinal cells;
a means for delivering the medicament to the retina, with said medicament having receptor sites; and
a means for fixing the antibodies to the receptor sites of the medicament to concentrate the medicament at the retinal cells.

15. The system of claim 14 wherein the administering means includes a device for performing an intra retinal injection.

16. The system of claim 14 wherein the administering means includes a device for performing an intra arterial injection.

17. The system of claim 14 wherein the delivering means includes a device for performing an intra retinal injection.

18. The system of claim 14 wherein the delivering means includes a device for performing an intra arterial injection.

19. The system of claim 14 wherein the medicament is selected from a group comprised of stem cells, melanin, and proteins.

20. The system of claim 14 wherein each antibody has a first receptor arm and a second receptor arm, and wherein the first receptor arms of the antibodies attach to the retinal cells and the second receptor arms of the antibodies attach to the receptor sites of the medicament.
